(19) United States
(12) Patent Application Publication
(10) Pub. No.: US 2013/0142830 A1
(43) Pub. Date: Jun. 6, 2013

(54) Tolerogenic Plasmacytoid Dendritic Cells Co-Expressing Cd8-Alpha And Cd8-Beta And Methods Of Inducing The Differentiation Of Regulatory T Cells Using Same

(71) Applicants: Omid AKBARI, Santa Monica, CA (US); Vincent LOMBARDI, Los Angeles, CA (US)

(72) Inventors: Omid AKBARI, Santa Monica, CA (US); Vincent LOMBARDI, Los Angeles, CA (US)

(73) Assignee: UNIVERSITY OF SOUTHERN CALIFORNIA, Los Angeles, CA (US)

(21) Appl. No.: 13/470,100

(22) Filed: May 11, 2012

Related U.S. Application Data
(60) Provisional application No. 61/486,221, filed on May 13, 2011.

(51) Int. Cl. C12N 5/0784

(52) U.S. Cl. CPC C12N 5/064
USPC 424/275.1; 435/355; 424/184.1; 435/34

(57) ABSTRACT

This invention discloses an unexpected discovery that plasmacytoid dendritic cells (pDCs) may be segregated into immunogenic or tolerogenic species based on novel biomarkers discovered herein. Exemplary biomarkers include CD8α⁺β⁺, CD8α⁺β⁻, CD8α⁻β⁻, C1q, and IL-9R. For example, pDCs with CD8α⁺β⁺, CD8α⁺β⁻ are tolerogenic and CD8α⁻β⁻ is immunogenic. Also disclosed are isolated pDCs, compositions comprising the pDCs, methods for isolating the pDCs, methods for treating immune-hyper-reactivity, such as airway hyper-reactivity, food allergy, asthma, and autoimmune disorders, by using compositions containing tolerogenic antigen presenting cells, preferably pDCs disclosed herein. Also disclosed are methods for identifying tolerogenic antigen presenting cells by using one or more novel biomarkers disclosed herein, including RALDH expression, CD8α, CD8βC1qa, C1qc, and IL-9R. Also disclosed are methods for inducing Treg cells by using the pDCs disclosed herein.

### CROSS-REFERENCE TO RELATED APPLICATIONS

This application claims the benefit of U.S. Provisional Application No. 61/486,221 filed on May 13, 2011. The above priority application is hereby incorporated herein by reference.

### STATEMENT REGARDING FEDERALLY SPONSORED RESEARCH AND DEVELOPMENT

This invention was made with government support under Grant No. ROI AI066020 awarded by the National Institutes of Health. The government has certain rights in the invention.

### FIELD OF THE INVENTION

The invention pertains to the field of ophthalmology. More particularly, the invention pertains to methods for acquiring and analyzing optical coherence tomography images to detect optic nerve diseases.

### BACKGROUND OF THE INVENTION

Dendritic Cells (DCs) constitute a family of cells with the unique ability to distinguish pathogens from innocuous microorganisms as well as self from non-self antigens'. These cells can further initiate a robust immune response against infectious agents or, in contrast, maintain immune tolerance to self-antigens. To accomplish these tasks, DCs are equipped with pattern recognition receptors which recognize motifs highly conserved in pathogens throughout the evolution². Engagement of these receptors triggers the up-regulation of co-stimulatory molecules and the production of immune mediators such as cytokines and chemokines. Along with the capacity of DCs to present antigens, these signals direct the differentiation of naïve CD4⁺ T cells into the appropriate subset of T helper cells (T_{H})^{3, 4}. Because they balance immunity and tolerance, DCs are considered as key regulators of the immune system^{1, 3}. However, it is not known in the art how DCs achieve these apparently opposite functions. Recent studies suggest that immunogenic and tolerogenic functions are assigned to different subpopulations of DCs^{1, 3, 4}, but the defining characteristics of these subpopulations of DCs have not yet been identified.

Subsets of tolerogenic DCs have been especially described in the guts and in the respiratory tract which are constantly in contact with dietary or airborne antigens respectively⁵⁻⁸. Because mucosas act as a barrier between the body and the environment, they are therefore continuously exposed to numerous harmless environnemental antigens. As a result, mucosal tissues are particularly prone to induce immune tolerance to innocuous antigens. For instance, the gut-associated lymphoid tissue possesses a subset of DCs with immuno-regulatory properties expressing the mucosal integrin CD103⁹. These cells are able to promote the differentiation of Foxp3⁺ T cells from naive CD4⁺ T cells. In the lungs, DCs sample airborne antigens as well as pathogens. It was demonstrated that, under normal conditions, respiratory exposure to antigen elicits the generation of IL-10-producing DCs resulting in immune tolerance¹⁰. Further studies suggested that plasmacytoid dendritic cells (pDCs) can be considered as mainly responsible for the maintenance of tolerance to allergens^{11, 12}. Indeed, their depletion in a murine model abolishes tolerance induction to inhaled antigens. In contrast, in some cases, innocuous airborne molecules such as antigens from pollens or house dust mites can be misinterpreted by DCs and considered as a danger. This results in the development of a Th2-driven allergic inflammation of the lungs^{4, 13}.

The capacity of DCs to maintain tolerance varies depending on the subset of DCs but also on the signals they received. DCs function can be modulated by various tolerogenic stimuli such as IL-10, 1,25-dihydroxyvitamin D3, Galectin-1 or interactions with apoptotic cells. IL-10-treated DCs display an immature phenotype, produce high amount of IL-10 and trigger the differentiation of regulatory T cells (Tregs) producing IL-10^{14, 15}. Similarly, 1,25-dihydroxyvitamin D3 enhance the tolerogenic properties of myeloid dendritic cells¹⁶. DCs that capture apoptotic cells acquire tolerogenic properties in order to mediate peripheral tolerance to self-antigens¹⁷. Recently Galectin-1, an endogenous glycan-binding protein, was described as capable to program DCs to become tolerogenic¹⁸.

As noted above, induction of tolerance is particularly important in mucosal tissues in terms of immune responses to antigens encountered in the respiratory and intestinal tracts. These sites are continuously exposed to a wide variety of environmental, nonpathogenic antigens, which induce hyper-reactivity or tolerance, rather than active immunity. That is, food allergen in intestinal tract or inhaled allergen in the airway generally do not induce protective immune responses. However, in individuals with allergenic asthma, processing of these protein antigens result in the induction of antigen-specific Th2-biasesed inflammatory responses that cause AHR and asthma. Therefore, it is desirable to have a better understanding of the specific events that led to AHR, which in turn will provide more effective therapeutic methods and/or pharmaceutical products to counter the hyper-reactivity.

### SUMMARY OF THE INVENTION

The present invention has unexpectedly discovered that pDCs can be segregated into three distinct populations according to their expression of surface markers CD8α or CD8α and CD8β. These subsets are not only different in phenotype but also functionally distinct since CD8α⁺β⁻ and CD8α⁺β⁺ pDCs are more potent inducers of CD4⁺ CD25⁺ Foxp3⁺ regulatory T cells (Tregs) compared to CD8α⁺β⁻ pDCs. Our findings indicate that, in a mouse model of allergic asthma, adoptive transfer of CD8α⁺β⁻ or CD8α⁺0 pDCs prevents the development of airway hyper-reactivity. In contrast, adoptive transfer of CD8α⁻β⁻ pDCs triggered sensitization in naive mice, indicating that CD8α⁺β⁺/CD8α⁺β⁻ pDCs and CD8α⁻β⁻ pDCs act in opposite directions. Therefore, CD8α⁻β⁻ pDCs represent a pro-inflammatory subpopulation of pDCs while CD8α⁺β⁺ can be considered as a tolerogenic subset.

By comparing the gene expression profile of the three subsets of pDCs described in the present invention, we found that the expression of CD98hc, a receptor for the Galectin-3, was significantly up-regulated in both CD8α⁺β⁻ and CD8αβ⁺ subsets. Adding Galectin-3 to sorted CD8α⁺β⁻ or CD8α⁺β⁺ pDCs in vitro, enhances the conversion of naive CD4⁺ T cells into Tregs.

In addition, it was also discovered that retinaldehyde dehydrogenase (RALDH) were up-regulated in tolerogenic pDCs. In particular, it was discovered that in conventional DCs, only two of the three RALDH isoforms were expressed (RALDH 1 and 2), whereas in tolerogenic pDCs, all three isoforms of RALDH (RALDH 1, 2, and 3) were up-regulated.

Thus, the present invention has unveil for the first time subsets of pDCs with the capacity to induce regulatory functions that may contribute to the establishment of immunological tolerance. These subsets are not only phenotypically but also functionally distinct as CD8α⁺β⁺ pDCs are more able to induce Foxp3⁺ Tregs than CD8α⁺β⁻ or CD8α⁻β⁻ pDCs. As demonstrated in the mouse model, the ability of the adoptively transferred tolerogenic pDCs to prevent the development of airway hyper-reactivity is due to their strong ability to induce CD4⁺ CD25⁺ Foxp3⁺ regulatory T cells in the lungs and periphery. That is, the tolerogenic pDCs of the present invention strongly support the differentiation of Foxp3⁺ CD4⁺ Tregs cells both in vivo and in vitro.

According, a first aspect of the present invention is directed to isolated pDCs selected from the group consisting of CD8α⁻β⁻, CD8α⁺β⁺, CD8α⁺β⁻ and a combination of CD8α⁺β⁺ and CD8α⁺β⁻. Embodiments in accordance with this aspect of the invention will generally include one or more isolated pDCs. In a preferred embodiment, the isolated pDCs is comprised essentially of one of the three subtypes selected from CD8α⁺β⁻, CD8α⁺β⁺, CD8α⁺β⁻. In another preferred embodiment, the isolated pDCs is comprised essentially of CD8α⁺β⁺ and CD8α⁺β⁻ in any proportion.

A second aspect of the present invention is directed to a composition comprising a population of tolerogenic or immunogenic pDCs. Embodiments in accordance with this aspect of the invention will either include tolerogenic pDCs or immunogenic pDCs. Tolerogenic pDCs are isolated pDCs expressing the surface marker CD8α, and may optionally express the surface marker CD8β. Immunogenic pDCs are pDCs that does not express CD8α or CD8β. Preferably, in the case of tolerogenic pDCs, the composition may further include TGF-β. More preferably, the composition may further include Galectin-3. In the case of immunogenic pDCs, the composition may preferably include an inhibitor of RALDH such as DEAR or any other suitable RALDH inhibitor known in the art.

A third aspect of the present invention is directed to a method for isolating or purifying a pDC. Methods in accordance to this aspect of the invention will generally include the steps of enriching pDC from a source; and sorting pDC into subtypes according to their surface marker. Preferably according to their CD8 subtypes as described above.

A forth aspect of the present invention is directed to a method of preventing inflammation or immune hyper-reactivity in a subject. Methods in accordance with this aspect of the invention will generally include the step of loading a tolerogenic pDC with an antigen; and administering the loaded pDC to the subject. In a preferred embodiment, the tolerogenic pDC is one selected from the group consisting of CD8α⁺β⁺, CD8α⁺β⁻, and a combination thereof.

A fifth aspect of the present invention is directed to a method for inducing the conversion of Foxp3+ regulatory T cells. Methods in accordance with this aspect of the invention will generally include the steps of bringing a tolerogenic antigen presenting cell into fluid communication with a CD4⁺ naïve T cell. Preferably, the antigen presenting cell is a tolerogenic pDC. In some preferred embodiments, the antigen presenting cell is pre-loaded with an antigen. More preferably, the CD4+naïve T cell and the antigen presenting cells are brought together in the presence of TGF-β, galectin-3, or both.

A sixth aspect of the present invention is directed to a method for modulating immune response in a subject who is suffering from immune hyper-reactivity or in need of boosting immune response. Methods in accordance with this aspect of the invention will generally include the steps of administering a composition to the subject, wherein said composition includes tolerogenic pDC or immunogenic pDC, depending on whether the subject is in need of suppressing or boosting an immune response against an antigen.

A seventh aspect of the present invention is directed to a method for identifying a tolerogenic antigen presenting cell. Methods in accordance with this aspect of the invention will generally include the steps of determining the expression levels of RALDH1, RALDH2, and RALDH3 in the antigen presenting cell; and designating the antigen presenting cell as tolerogenic if all three RALDHs are up-regulated compare to a reference.

Other aspects and advantages of the present invention will become apparent from the following detailed description and the appended claims.

### BRIEF DESCRIPTION OF THE DRAWINGS

FIG. 1 shows that plasmacytoid DCs express either CD8α or CD8α and CD8β. (a) Flow cytometry analysis of CD8α and CD813 surface expression on pDCs. Splenocytes and cells prepared from lymph nodes or lungs were stained with anti-IA/IE, anti-BST2 (clone 120G8), anti-CD8α and anti-CD813 antibodies. Plasmacytoid DCs were gated according to their co-expression of BST2 and IA/IE. Gates were set based on isotype controls, numbers in outlined areas indicate the percentage of positive cells in the designated region. (b) Confocal fluorescent microscopy of the expression of CD8α and CD8β at the surface of pDCs. Magnetically purified pDCs were stained with either anti-BST2, anti-IA/IE or anti-CD11c along with CD8α and CD8β antibodies. Original magnification ×1000. (c) Percentage of CD80α⁻ CD8β⁻ pDCs, CD8α⁺ CD8β⁻ pDCs and CD8α⁺ CD8β⁺ pDCs in lymph nodes, spleen, lungs and blood. Data are from two independent experiments (mean and SEM of two mice). (d) Surface expression of Siglec-H, Ly6C, B220, Ly49Q, IA/IE, CD80, CD86 and CD40 was assessed by flow cytometry on CD8α⁻ CD8β⁻ pDCs, CD8α⁺ CD8β⁻ pDCs and CD8α⁺ CD8β⁺ pDCs from peripheral lymph nodes. Shaded histograms represent isotype control antibodies; solid black lines specific stainings. Data are representative of three independent experiments.

FIG. 2 shows that CD8α and CD8β are co-expressed on a subset of pDCs but not on cDCs. (a) Expression of CD8α and CD8β was analyzed by flow cytometry on cDCs (CD11c^{high} BST2⁻) and pDCs (CD11c^{dim} BST2⁺) from pooled peripheral lymph nodes of Flt3L-treated BALB/c mice. Results are representative of two similar experiments. Numbers beside outline areas indicate percent of positive cells. (b) CD8α⁻ CD8β⁻, CD8α⁺ CD8β⁻ and CD8α⁺ CD8β⁺ pDCs or CD8α⁻ and CD8α⁺ cDCs were sorted by flow cytometry and total RNAs were subsequently isolated. Relative gene expression of CD8α and CD8β genes was assessed by quantitative real-time PCR. CD8α⁻ CD8β⁻ pDCs were used as a calibrator to evaluate CD8α and CD8β gene expression in CD8α⁺ CD8β⁻ and CD8α⁺ CD8β⁺ pDCs while CD8α⁻ mDCs served as a reference to measure CD8α and CD8β gene expression in CD8α⁺ mDCs. Data are the average±SEM of six independent experiments. (c) CD8α and CD8β surface expression was assessed in B2m KO mice lacking CD8⁺ T cells. Plasmacytoid DCs from pooled lymph nodes expanded by Flt3L-secreting B16 melanoma in WT or B2m KO C57BL/6 mice were stained with CD8α and CD8β antibodies. Gates were set on the basis of isotype controls and numbers in outlined areas represent the percentage of positive cells for each population. Data are representative of two experiments. (d) Expression of CD8α and CD8β was confirmed at the gene expression level by real-time PCR in CD8α⁺ CD8β⁻ and CD8α⁺ cD8β⁺ pDCs or CD8α⁻ and CD8α⁺ cDCs isolated by cell sorting from Flt3L-treated B2m KO mice. Data are the mean±SEM of three different experiments.

FIG. 3 shows that CD8α⁺ CD8β⁺ plasmacytoid dendritic cells express higher level of costimulation markers upon TLR stimulation but produce less cytokines. (a) Plasmacytoid DCs were isolated by magnetic separation from lymph nodes of Flt3L-treated mice. The surface expression of the costimulation molecules CD80 and CD86 was assessed on CD8α⁻ CD8β⁻ pDCs, CD8α⁺ CD8β⁻ pDCs and CD8α⁺ CD8β⁺ pDCs subtypes after 18 hours of stimulation with R848 (10 μg/ml) or CpG (10 μM). Data are representative of three similar experiments. Shaded histograms represent isotype control antibodies; dashed lines, specific staining of untreated cells; solid black lines, specific staining of CpG treated cells and solid grey lines, specific staining of R848 treated cells. (b) Percentages of CD8α⁻ CD8β⁻, CD8α⁺ CD8β⁻ and CD8α⁺ CD8β⁺ pDCs after 18 hours of stimulation with either medium, R848 (10 μg/ml) or CpG (10 μM). (c) Plasmacytoid DCs subsets were sorted by flow cytometry according to their expression of CD8α alone or in combination with CD8β. CD8α⁻ CD8β⁻ pDCs, CD8α⁺ CD8β⁻ pDCs and CD8α⁺ CD8β⁺ pDCs were subsequently stimulated with R848 (10 μg/ml) or CpG (10 μM) for 18 hours. Culture supernatants were tested for IFN-α and IL-10 by ELISA. Results are average±SEM of two independent experiments. (d) Plasmacytoid DCs were isolated by magnetic separation from peripheral lymph nodes of Flt3L-treated BALB/c mice and cultured for 60, 120 or 180 minutes in presence of OVA-APC (10 μg/ml) at 37° C. or 4° C. Cells were then washed and stained with CD8α and CD8β⁻ antibodies, APC fluorescence was analyzed by flow cytometry in the CD8α⁻ CD8β⁻ pDCs, CD8α⁺ CD8β⁻ pDCs or CD8α⁺ CD8β⁺ pDCs subpopulations. Gates were set according to relevant isotype. Data are average (mean fluorescence intensity (MFI) at 37° C.)−(MFI at 4° C.)±SEM of three separate experiments. (d) CD4⁺ T cells from DO11.10 mice were co-cultured with CD8α⁻ CD8β⁻, CD8α⁺ CD8β⁻ or CD8α⁺ CD8β^{- pDCs sorted from pooled peripheral lymph nodes of Flt}3L-treated mice. Cells were cultured for three days at a 1:10 ratio (pDCs:CD4⁺ T cells) with or without 10 μg/ml of OVA before being pulsed for 18 hours with ³H thymidine. The amount of radioactivity related to the number of cells was evaluated in a scintillation counter. (e) Simultaneously, supernatants were collected and tested for IL-2 cytokine by ELISA. Results are the mean of triplicates±SEM of one representative experiment out of two.

FIG. 4 shows that CD8α⁺ CD8β⁺ and CD8α⁺ CD8β⁻ loaded with OVA do not promote the development of airway hyperreactivity. (a) CD8α⁻ CD8β⁻ pDCs, CD8α⁺ CD8β⁻ pDCs, CD8α⁺ CD8β⁺ pDCs isolated by cell sorting or BM-DCs were loaded with OVA (10 μg/ml) for 4 hours. Cells were then adoptively transferred into naïve BALB/c mice (2×10⁵ cells per mice). Seven days after transfer, mice were challenged by intranasal administration of OVA (50 μg in 50 μl). (b) Subsequently, airway hyperresponsiveness was assessed by measurement of lung resistance, dynamic compliance and (c) Penh. Data are average±SEM of groups of 5 mice. (d) Representative lung histology of mice from panel (c). Lung tissue from mice transferred with CD8α⁻ CD8β⁻ pDCs, CD8α⁺ CD8β⁻ pDCs, CD8α⁺ CD8β⁺ pDCs or BM-DCs were stained with hematoxylin and eosin (H&E, upper panel) and periodic acid Schiff (PAS, lower panel). Arrows show the release of the mucus in the lumen. Original magnification x200, inset x600.

FIG. 5 shows that CD8α⁺ CD8β⁺ and CD8α⁺ CD8β⁻ pDCs prevent the development of airway hyperreactivity. (a) CD8α⁻ CD8β⁻, CD8α⁺ CD8β⁻ and CD8α⁺ CD8β⁺ pDCs isolated by cell sorting were loaded with OVA (10 μg/ml) for 4 hours. Cells were then adoptively transferred into naïve BALB/c mice (5×10⁵ cells per mice). Seven days after transfer, mice were immunized by intra-peritoneal injection of OVA (50 μg) in Alum (40 mg) and challenged at days 14, 15 and 16 by intranasal administration of OVA (50 μg in 50 μl saline). (b) At day 17, airway hyperresponsiveness was assessed by measurement of lung resistance, dynamic compliance. Results are the mean±SEM of 5 mice groups. (c) Representative lung histology of mice from panel (b). Lung tissue from mice transferred with CD8α⁻ CD8β⁻ pDCs, CD8α⁺ CD8β⁻ pDCs, CD8α⁺ CD8β⁺ pDCs or saline were stained with hematoxylin and eosin (H&E, upper panel) and periodic acid Schiff (PAS, lower panel). Arrows show the release of the mucus in the lumen. Original magnification ×200, inset ×600.

FIG. 6 shows that CD8α⁺ CD8β⁺ and CD8α⁺ CD8β⁻ pDCs promote the conversion of naive CD4⁺ T cells into CD4⁺ CD25⁺ Foxp3⁺ T cells in vivo. Subsets of CD8α⁻ CD8β⁻ pDCs, CD8α⁺ CD8β⁻ and CD8α⁺ CD8β⁺ pDCs were sorted from lymph nodes of Flt3L-treated mice, loaded with OVA and co-transferred by intravenous injection with OVA-specific CD4⁺ T cells (3×10⁵ pDCs and 3×10⁶ CD4⁺ T cells). Four days later, mice were challenged by intranasal administration of OVA (50 μg). At day 5, spleen and lymph node were harvested and expression of Foxp3 was analyzed by flow cytometry in OVA-specific T cells. Cells were gated on CD4⁺ KJ1.26⁺ CD25⁺ T cells. Data are representative of two independent experiments.

FIG. 7 shows that CD98hc is overexpressed in CD8α⁺ CD8β⁻ pDCs and CD8α⁺ CD8β⁺ pDCs compared to CD8α⁻ CD8β⁻ pDCs. (a) Gene expression profile of CD8α⁺ CD8β⁻ pDCs and CD8α⁺ CD83⁺ pDCs sorted from pooled peripheral lymph nodes was evaluated by microarray analysis. Numerical data represent the relative gene expression compared to CD8α⁻ CD8β⁻ pDCs. (b) Analysis of the gene expression of CD8α, CD8β and CD98hc by real-time PCR in CD8α⁻ CD8β⁻ pDCs, CD8α⁺ CD8β⁻ and CD8α⁺ CD8β⁺ pDCs isolated from peripheral lymph nodes. Results presented are the mean±SEM of six independent experiments. (c) Flow cytometry of the surface expression of CD98hc on CD8α⁻ CD8β⁻ pDCs, CD8α⁺ CD8β⁻ and CD8α⁺ CD8β⁺ pDCs. Results are the average of MFI±SEM of three different experiments.

FIG. 8 shows that Galectin-3 increases the conversion of naïve CD4⁺ T cells into CD4⁺ CD25⁺ Foxp3⁺ T cells by CD8α⁺ CD8β⁺ and CD8α⁺αCD8β⁻ pDCs. (a) Flow cytometry of intracellular expression of Foxp3 in CD4⁺ T cells cultured with CD8α⁻ CD8β⁻ pDCs, CD8α⁺ CD8β⁻ or CD8α⁺ CD8β⁺ pDCs. Sorted CD8α⁻ CD8β⁻, CD8α⁺ CD8β⁻ or CD8α⁺ CD8β⁺ pDCs were preincubated 12 hours with either medium or Galectin-3 (10 μg/ml). Cells were then washed and co-cultured with OVA-specific CD4⁺ T cells at a 1:10 ratio (pDC:T CD4⁺) for 5 days in presence of either OVA₃₂₃₋₃₃₉ (1 μg/ml) and/or TGF-β (1 ng/ml) as indicated. Cells were subsequently stained with CD3, CD4 and CD25 antibodies fixed, permeabilized and stained with an anti-Foxp3 antibody before flow cytometry analysis. (b) Flow cytometry of the intracellular production of IL-10 by CD4⁺ CD25⁺ Foxp3⁺ T cells. Alternatively, cells were restimulated with plate-bound α-CD3 for 4 hours with the last 2 hours in presence of Brefeldin A and subsequently permeabilized and stained with IL-10 specific antibody. Numbers in outlined areas indicate the percent of cells in the designated area. Data are representative of three experiments with comparable results.

FIG. 9 shows that for CD8α and CD8β staining of pDCs, quadrants were adjusted according to isotypic controls. “Fluorescence minus one” controls (i.e. CD8α staining versus isotype corresponding to CD8β antibody and CD8β staining versus isotype corresponding to CD8α) were performed to assess the proper correction of spectral overlaps.

FIG. 10 shows that the purity of pDCs subsets isolated by cell sorting. CD8α⁻ CD8β⁻ pDCs, CD8α⁺ CD80⁻ pDCs, CD8α⁺ CD80⁺ pDCs were confirmed to be >95% pure after post sorting reanalysis.

FIG. 11 CD8α⁺β⁻ and CD8α⁺β⁺ plasmacytoid dendritic cells (pDCs) exhibit high retinal dehydrogenase (RALDH) activity and promote the differentiation of CD4⁺ CD25⁺ Foxp3⁺ T cells in vitro in a transforming growth factor-β (TGF-β)- and retinoic acid-dependent manner. (a) Gene expression of Aldhla1 Aldhla2, and Aldhla3 was assessed by real-time PCR in CD8α⁻β⁻, CD8α⁺β⁻, and CD8α⁺β⁺ pDCs from peripheral lymph nodes or in CD103⁻ and CD103⁺ DCs from mesenteric lymph nodes of Fms-like tyrosine kinase 3-ligand (Flt3-L)-treated mice. Data are mean±s.e.m. of two independent experiments. P-values were calculated with Student's t-test. *P-value <0.05 (CD8α⁺β⁻ pDC and CD8α⁺β⁺ pDC compared to CD8α⁻β⁻). ^{#}P-value <0.05 (CD103⁺ cDC compared to CD103⁻ cDC). (b) pDCs were incubated for 45 min at 37° C. in the presence of Aldefluor substrate with or without diethylaminobenzaldehyde (DEAB, RALDH inhibitor) to determine background staining. Cells were subsequently stained with anti-CD11c, anti-mPDCA1, anti-CD8α, and CD8αβ antibodies, and analyzed by flow cytometry to detect RALDH activity. Values are average of (MFI without RALDH inhibitor) (MFI with RALDH inhibitor)±s.e.m. of three separate experiments. (c) Naïve ovalbumin (OVA)-specific CD4⁺ T cells were cultured with CD8α⁻β⁻, CD8α⁺β⁻, or CD8α⁺β⁺ pDCs at a 1:10 ratio (pDC:T CD4⁺) for 5 days in the presence of OVA 323-339 peptide with or without TGF-β(1 ng ml⁻¹) or LE540 (1 μM) as indicated. Cells were subsequently stained with CD3, CD4, and CD25 antibodies, fixed, permeabilized, and stained for intracellular Foxp3 before flow cytometry analysis. Foxp3 expression was analyzed among CD3⁺ CD4⁺ CD25⁺ cells; dot plots are representative of three independent experiments. P-values were calculated with Student's t-test. *P-value <0.05 (CD8α⁺β⁻ and CD8α⁺β⁺ compared to CD8α⁻β⁻).

FIG. 12 shows the expression pattern of surface markers in murine pDCs.

FIG. 13 shows the co-expression pattern of Galetin-3 and its receptor CD98hc on murine pDC subsets. *p-value <0.01.

FIG. 14 shows the co-expression pattern of C1qa and C1qc in murine tolerogenic pDCs.

FIG. 15 shows the identification of tolerogenic pDC in human using C1qa and C1qc antibodies. That is C1qa⁺c⁺ pDC is a tolerogenic pDC.

FIG. 16 shows the identification of tolerogenic pDC in human using IL-9R specific antibodies. That is IL-9R⁺ pDC is a tolerogenic pDC.

### DETAILED DESCRIPTION

Unless otherwise indicated, all terms used herein have the meanings consistent with same meaning that the terms have to those skilled in the art of the present invention. It is to be understood that this invention is not limited to the particular methodology, protocols, and reagents described, as these may vary.

As used herein the terms CD8 refers to cluster of differentiation 8 co-receptor. CD8 is a transmembrance glycoprotein that serve as a co-receptor for T cell receptor. It has two isoforms CD8α and CD8β.

As used herein, the term “CD8α⁻β⁻ pDC” refers to plasmacytoid dendritic cell expressing neither CD8α nor CD8β.

As used herein, the term “CD8α⁺β⁺ pDC” refers to plasmacytoid dendritic cell expressing both CD8α and CD8β.

As used herein, the term “CD8α⁺β⁻ pDC” refers to plasmacytoid dendritic cell expressing CD8α but not CD8β.

As used herein, the term “C1qa⁺c⁺ pDC” refers to plasmacytoid dendritic cell expressing both C1qa and C1qc.

As used herein, the term “IL-9R⁺ pDC” refers to plasmacytoid dendritic cell expressing IL-9R.

Amongst other aspects of the present invention, certain embodiments and/or findings in accordance with the present invention include:

1. Plasmacytoid dendritic cells expressing CD8α alone or combined with CD8β;
2. CD8α⁻β⁻, CD8α⁺β⁻ and CD8α⁺β⁺ pDCs present distinct cytokine production, antigen uptake and priming capacities;
3. Transfer of CD8α⁻β⁻ pDCs triggers the development of airway inflammation;
4. Transfer of CD8α⁺β⁺ pDCs or CD8α⁺β⁻ prevents the development of airway inflammation;
5. CD8α⁺β⁺ pDCs promote the differentiation of CD4⁺ CD25⁺ Foxp3⁺ T cells in vivo;
6. CD8α⁺β⁺ pDCs and CD8α⁺β⁻ pDCs overexpressed CD98hc; and
7. CD8α⁺β⁺ pDCs and CD8α⁺β⁻ pDCs promote the differentiation of Foxp3⁺ CD4⁺ T cells in vitro in a TGF-β and Galectin-3-dependent manner as well as in a RADLH-dependent manner.
8. Tolerogenic pDCs in mice express CD8α and/or CD8β, and also C1qa, C1qc and IL-9R. Thus, C1qa⁺, C1qc⁺ and IL-9R⁺ may also serve as biomarkers to identify tolerogenic pDCs.
9. Human pDCs do not express CD8α or CD8β, therefore, C1qa⁺, C1qc⁺ and IL-9R⁺ are the characterizing biomarkers for identifying tolerogenic pDCs in human.

According, a first aspect of the present invention is directed to isolated pDCs selected from the group consisting of CD8α⁻β⁻, CD8α⁺β⁺, CD8α⁺β⁺, C1qa⁺, C1qc⁺, IL-9R⁺, a combination of CD8α⁺β⁺ and CD8α⁺β⁻, and a combination of C1qa⁺, C1qc⁺ and IL-9R⁺. Embodiments in accordance with this aspect of the invention will generally include one or more isolated pDCs. In a preferred embodiment, the isolated pDCs is comprised essentially of one of the three subtypes selected from CD8α⁻β⁻, CD8α⁺β⁺, CD8α⁺β⁻. In another embodiment, the isolated pDCs is human pDCs expressing C1qa, C1qc and/or IL-9R. In yet another preferred embodiment, the isolated pDCs is comprised essentially of CD8α⁺β⁺ and CD8α⁺β⁻ in any proportion.

An “isolated” pDC is a pDC that is found in a condition other than its native environment, such as apart from blood and animal tissue. In a preferred form, the isolated pDC is substantially free of other cells and tissues, particularly other cells of animal origin. The term “purified CD8α⁻β⁻ pDCs” means a composition having CD8α⁻β⁻ pDCs with no population, or decreased population of CD8α⁺β⁺ pDCs or CD8α⁺β⁻ pDCS as described herein. The other purified pDCs are defined analogously. It is preferred to provide the “purified pDCs” in a highly purified form, i.e. greater than 95% pure, more preferably greater than 99% pure.

A second aspect of the present invention is directed to a composition comprising a population of tolerogenic or immunogenic pDCs. Embodiments in accordance with this aspect of the invention will either include tolerogenic pDCs or immunogenic pDCs. Tolerogenic pDCs are isolated pDCs expressing the surface marker CD8α, and may optionally express the surface marker CD8β. In human pDCs, they are pDCs that express C1qa, C1qc, and/or IL-9R. Immunogenic pDCs are pDCs that do not express CD8α or CD8β. In humans, they are pDCs that do not express any of C1qa, C1qc, or IL-9R. Preferably, in the case of tolerogenic pDCs, the composition may further include TGF-β. More preferably, the composition may further include Galectin-3. In the case of immunogenic pDCs, the composition may preferably include an inhibitor of RALDH such as DEAB or any other suitable RALDH inhibitor known in the art. More preferably, the composition may further include a suitable carrier.

The term “carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

A third aspect of the present invention is directed to a method for isolating or purifying a pDC. Methods in accordance to this aspect of the invention will generally include the steps of enriching pDC from a source; and sorting pDC into subtypes according to their surface marker. Preferably according to their CD8 subtypes as described above, and in humans, according to their C1q and IL-9R subtype. In a preferred embodiment, the pDCs, cells were isolated by using antibody specific for pDCs such as anti-mPDCA-1 to label the pDCs and then positively sorted cells are sorted by magnetic sorting or flow cytometry into the purified subsets of pDCs. Having been described herein the existence of the subsets of pDCs and their immunogenic/tolerogenic properties, those skilled in the art will recognize that other methods of cell separation/extraction/purification known in the art may also be advantageously adapted to obtain the three purified pDCs.

A forth aspect of the present invention is directed to a method of preventing inflammation or immune hyper-reactivity in a subject. Methods in accordance with this aspect of the invention will generally include the step of loading a tolerogenic pDC with an antigen; and administering the loaded pDC to the subject. In a preferred embodiment, the tolerogenic pDC is one selected from the group consisting of CD8α⁺β⁺, CD8α⁺β⁻, and a combination thereof. In another preferred embodiment, the tolerogenic pDC is a human pDC selected from C1qa⁺c⁺ and IL-9R⁺. Those skilled in the art will recognize that any condition that may be treated by induction of regulatory T cells (e.g. organ transplant, allergies, autoimmune disorders, etc.) may benefit from methods in accordance with this aspect of the invention.

A fifth aspect of the present invention is directed to a method for inducing the conversion of Foxp3⁺ regulatory T cells. Methods in accordance with this aspect of the invention will generally include the steps of bringing a tolerogenic pDC within fluid communication with a CD4⁺ naïve T cell. The pDC is preferably one pre-loaded with an antigen. More preferably, the CD4⁺ naïve T cell and the tolerogenic pDC are brought together in the presence of TGF-β, Galectin-3, or both. Thus, these tolerogenic pDCs have the capacity to convert antigen specific T cells reacting to allergens such as house dust mite or Aspergillus to regulatory T cells and dampen the unwanted immune responses in patients.

A sixth aspect of the present invention is directed to a method for modulating immune response in a subject who is suffering from immune hyper-reactivity or in need of boosting immune response. Methods in accordance with this aspect of the invention will generally include the steps of administering a composition to the subject, wherein said composition includes tolerogenic pDC or immunogenic pDC, depending on whether the subject is in need of suppressing or boosting an immune response against an antigen. In a preferred embodiment, the subject is one suffering from asthma, Th2-driven airway inflammation, allergic diseases including food allergy and autoimmune diseases with unwanted or excessive T cell responses.

A seventh aspect of the present invention is directed to a method for identifying a tolerogenic antigen presenting cell. In some embodiments, methods in accordance with this aspect of the invention will generally include the steps of determining the expression levels of RALDH1, RALDH2, and RALDH3 in the antigen presenting cell; and designating the antigen presenting cell as tolerogenic if all three RALDHs are up-regulated compare to a reference. In other embodiments, methods in accordance with this aspect of the invention will generally include the steps of selecting a surface marker in a known tolerogenic pDC as a test biomarker; and testing an isolated pDC expressing the selected marker to determine its tolerogenic property.

While not intending to be limited by any particular theory, we offer the following discussion to further facilitate a complete understanding of the various ramifications of the present invention.

### Detailed Discussion of Certain Findings

Plasmacytoid Dendritic Cells Express CD8α Alone or Combined with CD8β

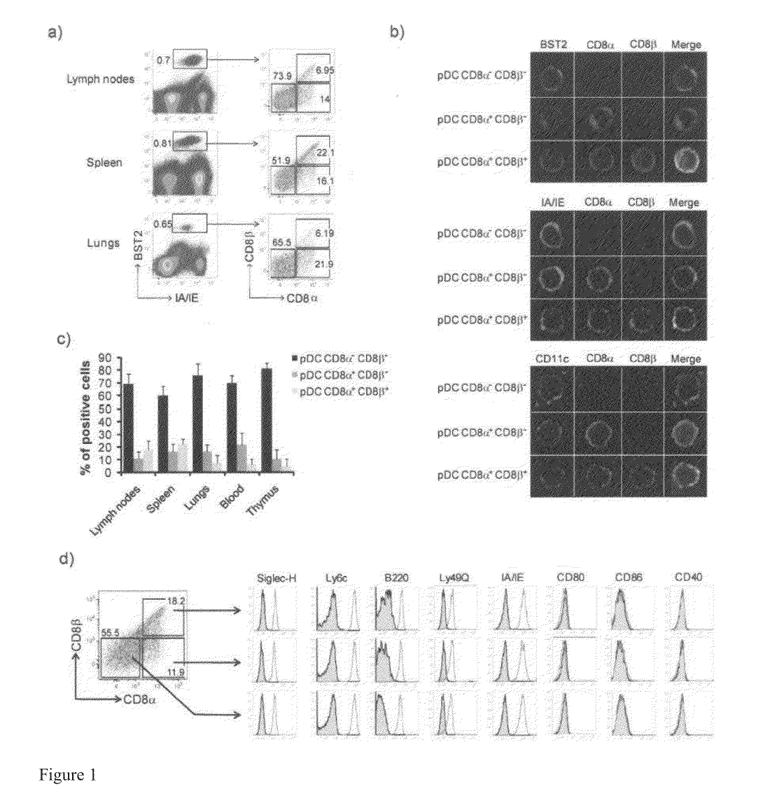

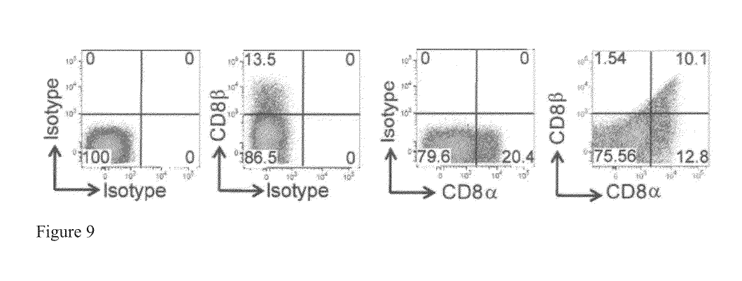

In a selection of organs (spleen, peripheral lymph nodes and lungs), we analyzed by flow cytometry the expression of an assortment of myeloid and lymphoid markers on pDCs defined by their expression of the bone marrow stromal antigen 2 (BST2), a specific marker of pDCs, and of MHC class II. We demonstrated for the first time that a fraction of pDCs can express either CD8α or both CD8α and CD8β (FIG. 1a). The dot plot obtained for the CD8α and CD8β staining suggests that CD8α and CD8β are expressed as a dimer. To confirm the accuracy of our analysis, we performed several additional control experiments with isotype controls (FIG. 9). Since the expression of CD8β has never been previously described on any subset of DCs, we used two different monoclonal antibodies specific for CD8β (H35-17.2 and 53-5.8) to evaluate the expression of CD8β on pDCs by flow cytometry. Similar results were obtained with both clones (Data not shown). To investigate further and validate the results with flow cytometry, we performed a series of experiments with confocal microscopy and analyzed the co expression of pDC markers (BST2, IA/IE and CD11c) with CD8α and CD8β on pDCs purified from peripheral lymph nodes (FIG. 1b). Our analysis with confocal microscopy, confirmed that a fraction of pDCs express CD8α, alone or along with CD8β (FIG. 1b). To evaluate the frequency of the three subsets among the pDC population, we analyzed the pDC population in lymph nodes, spleen, lungs, blood and thymus of naïve mice for the expression of CD8α and CD8β. Our data suggest that CD8α⁺β⁻ pDCs represent 10 to 22% of pDC repertoire, while CD8α⁺β⁺ pDCs are 4 to 23% of the whole population (FIG. 1c). The three subtypes of pDCs described herein exhibit all the specific markers of terminally differentiated pDCs (Siglec-H, Ly6C, B220 and Ly49Q) and display an immature phenotype with a low expression of co-stimulatory molecules CD40, CD80 and CD86 (FIG. 1d). Taken together, these data reveal that pDCs can be divided in three subpopulations: CD8α⁻β⁻, CD8α⁺β⁻ and CD8α⁺β⁺ pDCs.

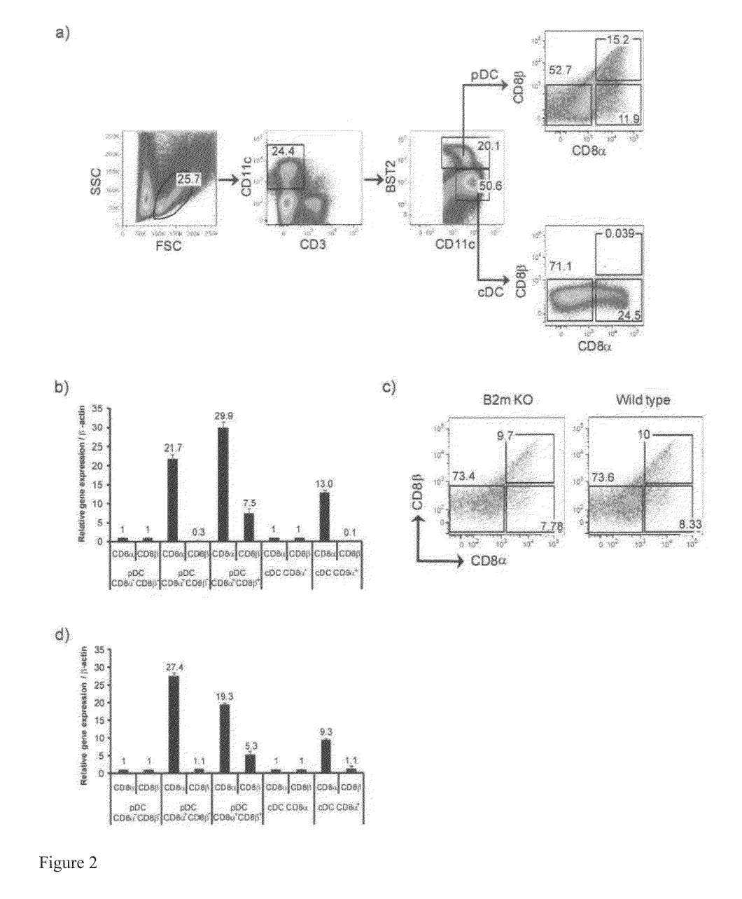

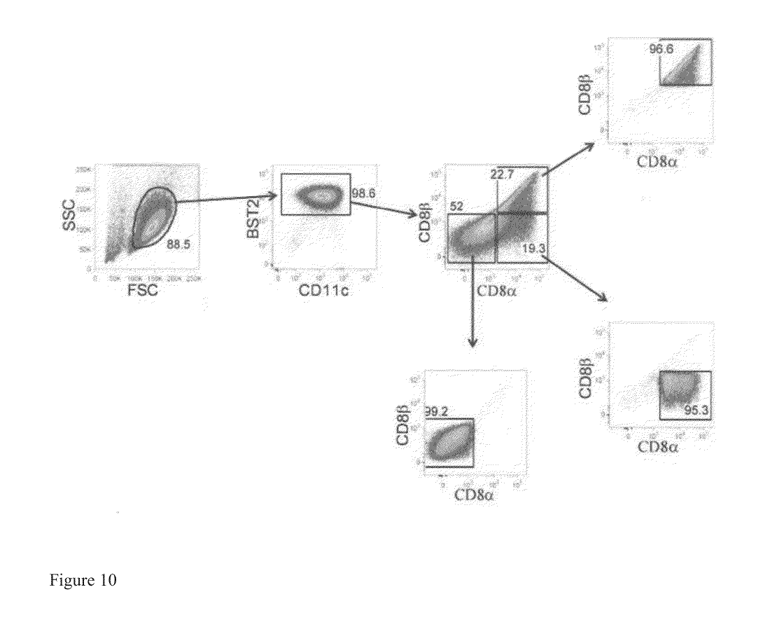

To test whether an in vivo expansion of the population of DCs affects the expression of CD8α or CD8β at the surface of pDCs, tumor cells expressing Flt3L were transferred into BALB/c mice. Flt3L acts on hematopoietic stem cells and controls their differentiation into DCs, this treatment expands the population of DCs by 15 to 20 fold after 14 days without activating the cells. We observed that Flt3L treatment does not significantly affect the level of expression of CD8α and CD8β on pDCs (FIG. 2a). The expression of CD8α and CD8β was assessed simultaneously on pDCs (CD11c⁺ BST2⁺ cells) and on conventional DCs (cDCs, CD11c⁺ BST2⁻ cells). At the surface of cDCs, only CD8α expression was detected (FIG. 2a). To prevent any irrelevant signal coming from CD8⁺-expressing T cells, DCs were gated on CD11c⁺ cells while T cells were excluded in the gating strategy by CD3 staining (FIG. 2a). These results were validated at the gene expression level after isolation of each population by cells sorting (cf. cell sorting purity in FIG. 10). Accordingly, with the surface phenotype, CD8α gene expression was detected in CD8α⁺β⁻ pDCs while both CD8α and CD8β genes were overexpressed in the CD8α⁺β⁺ subset (FIG. 2b). As expected CD8β gene expression was never detected in cDCs (FIG. 2b). Ultimately, we performed an analysis of CD8α and CD8β expression on DCs of mice lacking CD8⁺ T cells to prevent any uptake of CD8 antigens coming from CD8⁺ T cells. Thus, we used β-2 microglobuline (B2m) knockout mice lacking conventional CD8⁻ T cells and asked if they possess CD8α or CD8β pDC subsets. Our data from B2m knockout mice confirmed the presence of CD8α or CD8β pDC repertoire, similar to the level detected in the wild type mice (FIG. 2c). To further assess the expression of CD8α or CD8β at mRNA level, we performed real time quantitative PCR on the sorted subpopulation of pDCs from B2m knockout mice and confirmed the results obtained in FIG. 2c at the mRNA expression level (FIG. 2d). Collectively, these data confirm that three subpopulation of pDCs exist based on the expression of CD8α or co-expression of CD8α and CD8β.

CD8α⁻β⁻, CD8α⁺β⁺ and CD8α⁺β⁺ pDCs Present Distinct Cytokine Production, Antigen Uptake and Priming Capacities

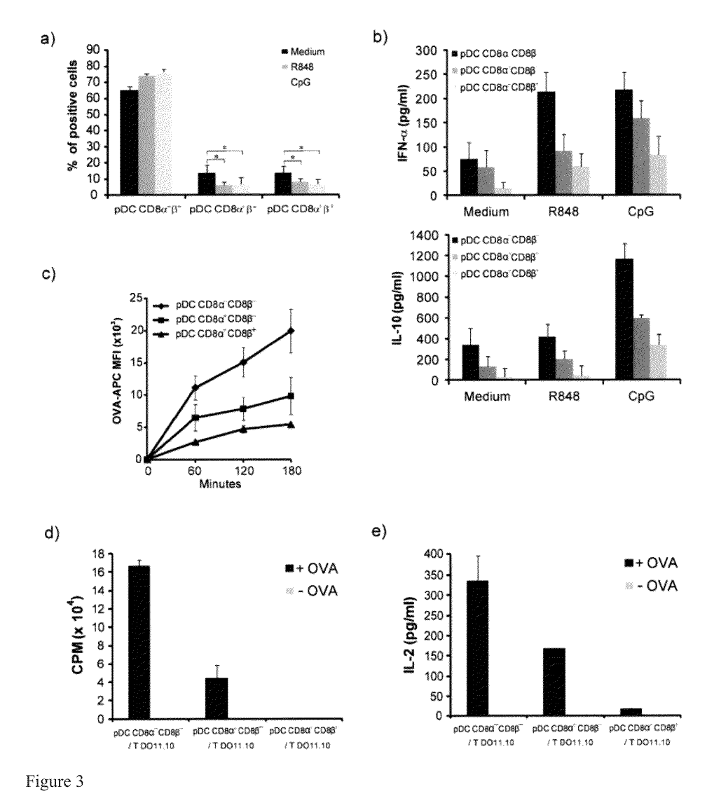

The main function of DCs is to prime naïve T cells by presenting antigen and providing additional signals through co-stimulatory molecules and production of cytokines. To address whether the populations of pDCs described herein differ in these functions, we stimulated them with TLR ligands and assessed the expression of co-stimulatory molecules along with the cytokine production. We stimulated CD8α⁻β⁻, CD8α⁺β⁻ and CD8α⁺β⁺ pDCs with R848 (synthetic TLR7 ligand) and CpG oligonucleotides (TLR9 ligand) and assessed the surface expression of CD80 and CD86 co-stimulation molecules as well as the production of IFN-α and IL-10. Plasmacytoid DCs are known to produce large amount of type I interferon in response to a viral infection but also to be potent inducer of immune tolerance by producing IL-10. We observed that, after engagement of either TLR7 or TLR9, CD8α⁺β⁺ pDCs and CD8α⁺β⁻ pDCs present a higher level of CD80 and CD86 compared to CD8α⁻β⁻ subset (data not shown). We also determine that following TLR7 or TLR9 stimulation, the expression of CD8α and CD8β decreases (FIG. 3a). Interestingly, it appeared that CD8α⁻β⁻ produce more IFN-α and IL-10 than CD8α⁺β⁻ and CD8α⁺β⁺ pDCs upon stimulation (FIG. 3b). To assess the capacity of pDC subsets in uptaking antigens, pDC subsets were incubated with ovalbumin-APC (OVA-APC) and tested for antigen uptake capacity at various time points. Our data suggest that CD8α⁺β⁺ pDCs have a high capacity to capture antigen whereas CD8α⁺β⁻ and CD8α⁻β⁻ pDCs present an inteimmediate and low uptake capacity respectively (FIG. 3c). We then examined whether the three subtypes of pDCs described in this study have different abilities to prime antigen specific CD4⁴ T cells. We performed an in vitro co-culture between these populations of pDCs and T cells isolated from DO11.10 mice with an OVA-specific transgenic TCR. In response to OVA, we observed a robust proliferation in co-culture performed with CD8α⁻β⁻ pDCs in compare with CD4+ T cells primed with either CD8α⁺β⁻ or CD8α⁺β⁺ pDCs (FIG. 3d). As expected, the production of IL-2 by CD4+ DO1110 cells was correlated with the proliferation assay performed in FIG. 3d (FIG. 3e). Thus our data suggest that pDC subsets segregated according to their expression of CD8α or CD813, present significant differences in their capacity to prime naïve CD4⁺ T cells and to produce cytokines or to capture antigens.

Transfer of CD8α⁻β⁻ pDCs Triggers the Development of Airway Inflammation

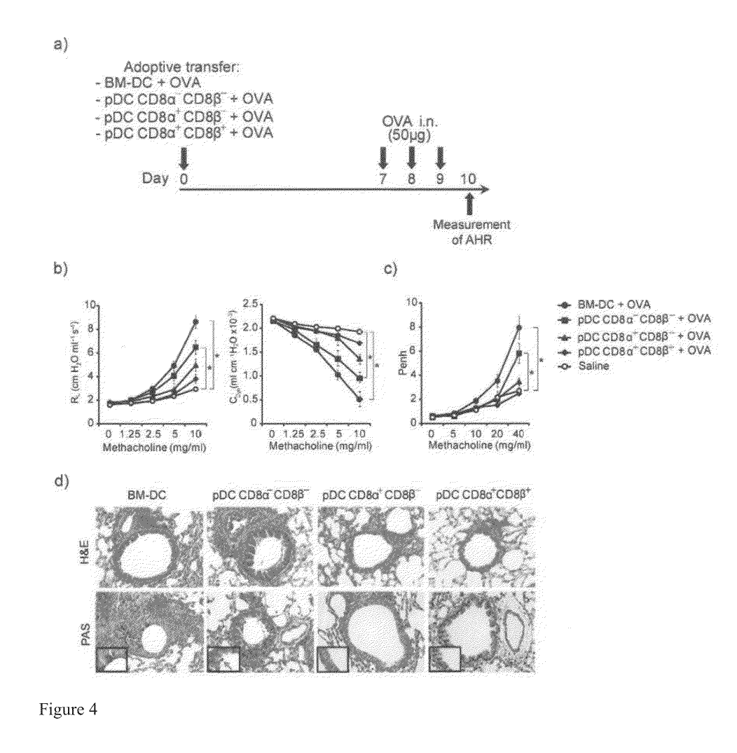

Taking into consideration the noticeable difference among the pDC subsets, we addressed the potency of CD8α⁻β⁻, CD8α⁺β⁻ or CD8α⁺β⁺ pDCs to trigger antigen sensitization in a mouse model of airway hyper-reactivity. We adoptively transferred the three subtypes of pDCs characterized in this study and compared the results with the recipients of bone marrow-derived DCs (BM-DCs). These cells were loaded for 4 hours with OVA and washed prior to the transfer. One week later, mice were challenged by three consecutive intranasal administrations of OVA (FIG. 4a). This model allows us to evaluate whether a subset of DCs is able to initiate sensitization to OVA. The lung inflammation was assessed by analysis of lung histology and the lungs function was evaluated by plethysmography. We showed that CD8α⁻β⁻ pDCs support the development of strong AHR measured as lungs resistance and dynamic compliance in anesthetized, tracheotomized and ventilated animals or as enhance pause (Penh) in conscious animals (FIGS. 4b and c). In contrast CD8ct⁺β⁻ pDCs trigger intermediate but not significant AHR while CD8α⁺β⁺ pDCs are nearly unable to induce AHR in the recipients (FIGS. 4b and c). We also examined lungs histology and stained lung sections with hematoxylin and eosin (H&E) to observe cellular infiltration or periodic acid Schiff (PAS) to examine mucus production. In accordance with the plethysmography results, a massive cell infiltration as well as an significant mucus production was observed in mice transferred with CD8α⁻β⁻ pDCs (FIG. 4d). In contrast, a minor cellular infiltration and no mucus secretion was observed after transfer of CD8α⁺β⁺ pDCs while lungs of mice which received CD8α⁺β⁺ pDCs did not present any abnormalities (FIG. 4d). These results collectively show that CD8α⁺β⁻ pDCs can be considered as an immunogenic population of pDCs in contrast with CD8α⁺β⁻ or CD8α⁺β⁺ pDCs which has the ability to regulate the immune responses in the lungs.

CD8α⁺β⁺ pDCs or CD8α⁺β⁻ Induce Mucosal Tolerance

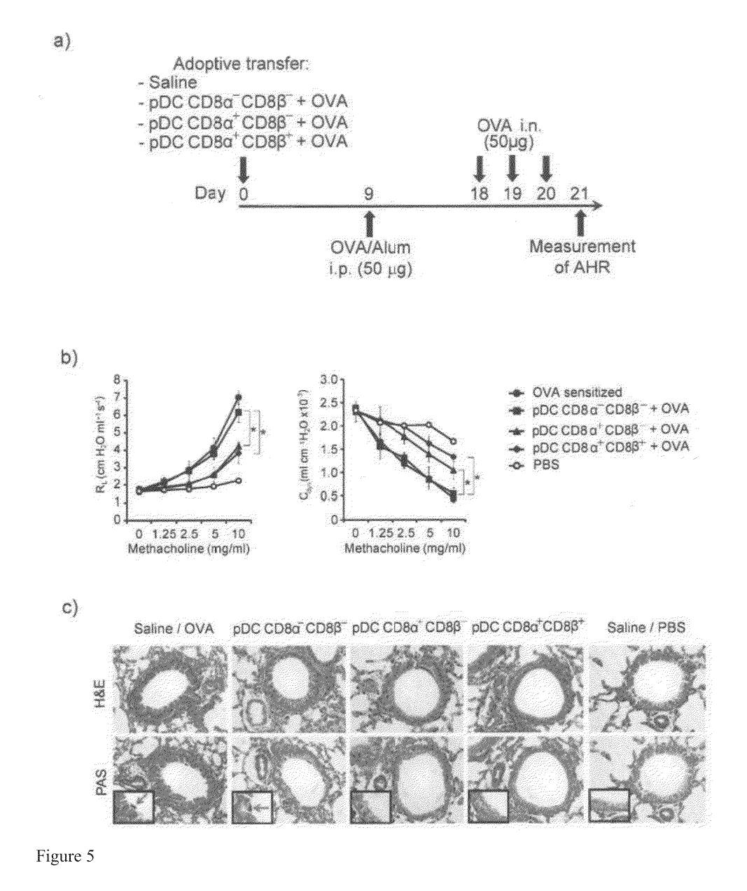

Alternatively, we tested the capacity of pDC subsets to induce mucosal tolerance and asked if pDC subsets had the capacity to transfer T cell unresponsiveness. Naïve mice were initially transferred with the different subsets of pDCs loaded with OVA prior intra-peritoneal injection of OVA in Alum followed by OVA intranasal challenges (FIG. 5a). Sorted pDC subsets from draining lymph nodes of mice exposed to OVA were adoptively transferred into naïve mice, which were subsequently immunized with OVA in alum. Adoptive transfer of tolerogenic DCs blocked subsequent OVA sensitization by immunization with OVA in alum; lung function results from recipient mice immunized with OVA showed lower lung resistance and higher dynamic compliance (FIG. 5b). Our results indicated that adoptive transfer CD8α⁺β⁻ pDCs and more especially CD8α⁺β⁺ pDCs blocked subsequent OVA sensitization by immunization with OVA in alum; as indicated by measuring lung resistance and dynamic compliance in the recipient mice (FIG. 5b). In contrast, animals which received CD8α⁻β⁻ pDCs showed severe AHR similar to the recipients of BM-DCs (positive control)(FIG. 5b). Examination of the lungs of mice that received CD8α⁺β⁻ or CD8α⁺β⁺ pDCs showed a reduction in airway inflammation, cellular infiltration and mucus production, indicating that CD8α⁺β⁻ or CD8α⁺β⁺ subset of pDCs can induce mucosal tolerance and had potent in vivo regulatory activity against asthma and Th2-driven airway inflammation (FIG. 5c).

CD8α⁺ CD8β⁺ pDCs Promote the Differentiation of CD4⁺ CD25⁺ Foxp3⁺ T Cells in Vivo

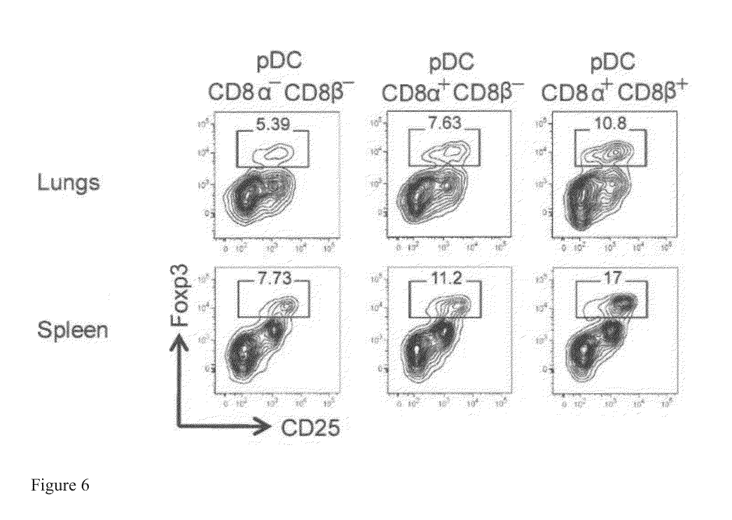

To investigate why CD8α⁺β⁻ pDCs and CD8α⁺β⁺ pDCs induce immune tolerance, we assessed whether these cells trigger the differentiation of CD4⁺ CD25⁺ Foxp3⁺ T cells, a phenotype characteristic of Tregs; cells that are often instrumental in immune tolerance mechanisms. In this regard, we co-transferred naïve CD4⁺ T cells isolated from OVA-specific DO11.10 mice with either CD8α⁻β⁻ pDC, CD8α⁺β⁺ pDCs or CD8α⁺β⁺ pDCs loaded with OVA. After four days, we challenged the mice intranasally with OVA on three consecutive days before analyzing Foxp3 expression in the spleen and in the lungs. We tracked the conversion of the cells we transferred into Foxp3-expressing cells using the clonotypic antibody KJ1.26 specific of the OVA-specific transgenic TCR. We observed an increased proportion of CD4⁺ CD25⁺ Foxp3⁺ DO11.10 T cells in mice which received CD8α⁺β⁻ pDCs and more especially CD8α⁺β⁺ pDCs (FIG. 6). With this last subset, the percentage of CD4⁺ CD25⁺ Foxp3⁺ T cells was increased by more than two fold in the lungs as well as the spleen. We demonstrated here that CD8α⁺β⁺ pDCs and CD8α⁺β⁻ favor the development of CD4⁺ CD25⁺ Foxp3⁺ T cells in vivo.

CD8α⁺ CD8β⁺ pDCs and CD8α⁺ CD8β⁻ pDCs Overexpressed CD98hc

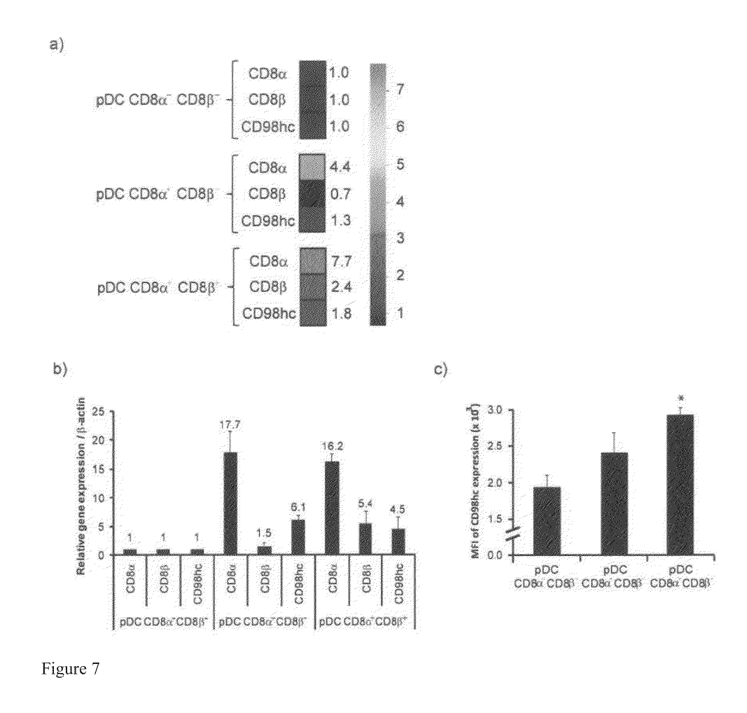

To understand why CD8α⁺β⁻ pDCs and CD8α⁺β⁺ pDCs present tolerogenic properties, we evaluated their gene expression profile by microarray analysis. It appeared that, compared to CD8α⁻β⁻ pDCs, CD98hc, a receptor for the Galectin-3, is selectively over expressed in CD8α⁺β⁻ and CD8α⁺β⁺ pDC subpopulation (FIG. 7a). The results obtained by microarray were confirmed several times by real-time PCR (FIG. 7b). Eventually, analysis of the surface expression of CD98hc by flow cytometry revealed that expression of CD98hc was up-regulated in CD8α⁺β⁻ subset and more particularly in CD8α⁺β⁺ pDCs (FIG. 7c).

Galectin-3 Promote the Differentiation Foxp3⁺ CD4⁺ T Cells by CD8α⁺β⁻ or CD8α⁺β⁺ pDCs

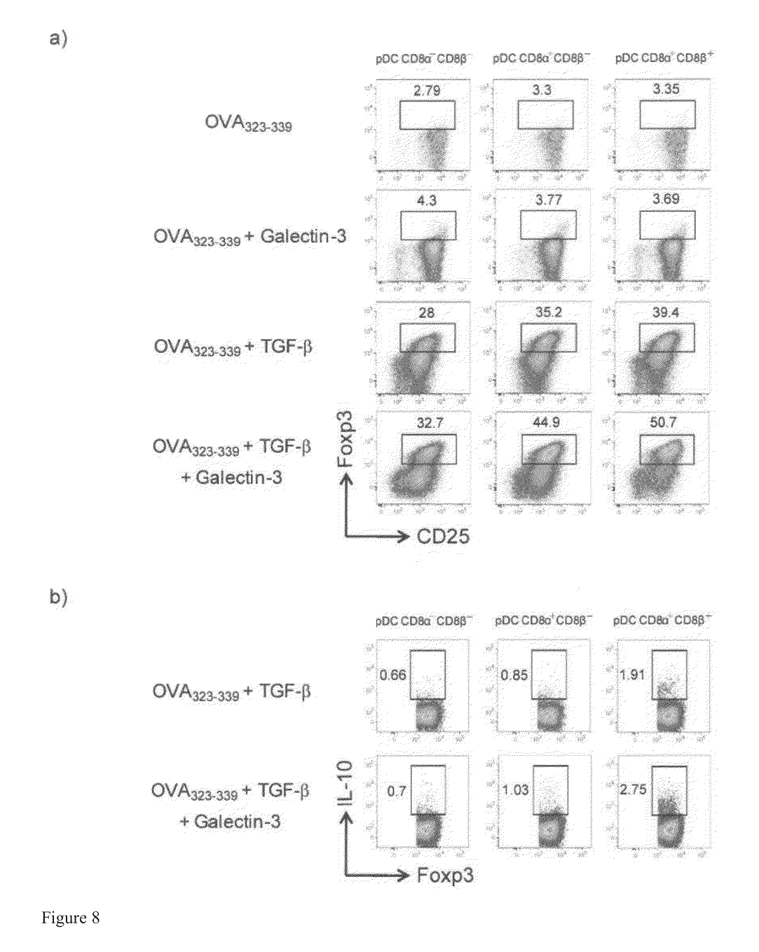

We then investigated the role of Galectin-3, the ligand for CD98hc, in induction of Tregs in vitro. Therefore, we pre-incubated pDC subsets with Galectin-3 prior to culturing them with OVA-specific naive CD4⁺ T cells in the presence of TGF-β, a cytokine indispensable to the development of Tregs. Similarly to the results obtained in vivo, we observed that, in the presence of TGF-β and after 5 days of culture, CD8α⁺β⁺ pDCs greatly elicit the development of Foxp3⁺ CD4⁺ T cells and, to a lesser extent for CD8α⁺β⁻ pDCs (FIG. 8a). Interestingly, Galectin-3 pre-incubation boost the capacity of pDCs to convert naïve CD4⁺ T cells into Foxp3-expressing cells especially for the CD8α⁺β⁻ pDCs and the CD8α⁺β⁺ subsets (FIG. 8a). We simultaneously analyzed the production of IL-10, an important regulatory cytokines, in Foxp3⁺ T cells. We observed that Tregs generated by CD8α⁺β⁺ pDCs produce more IL-10 than those differentiated by CD8α⁺β⁻ or CD8α⁻β⁻ pDCs (FIG. 8b). Altogether, these results show that CD8α⁺β⁻ pDCs and more particularly CD8α⁺β⁺ pDCs strongly support the development of IL-10 producing Foxp3⁺ CD4⁺ T cells in a TGF-β and a Galectin-3-dependent manner.

RALDII Expression in CD8α⁺β⁻ or CD8α⁺β⁺ pDCs is Responsible for Induction of Tregs

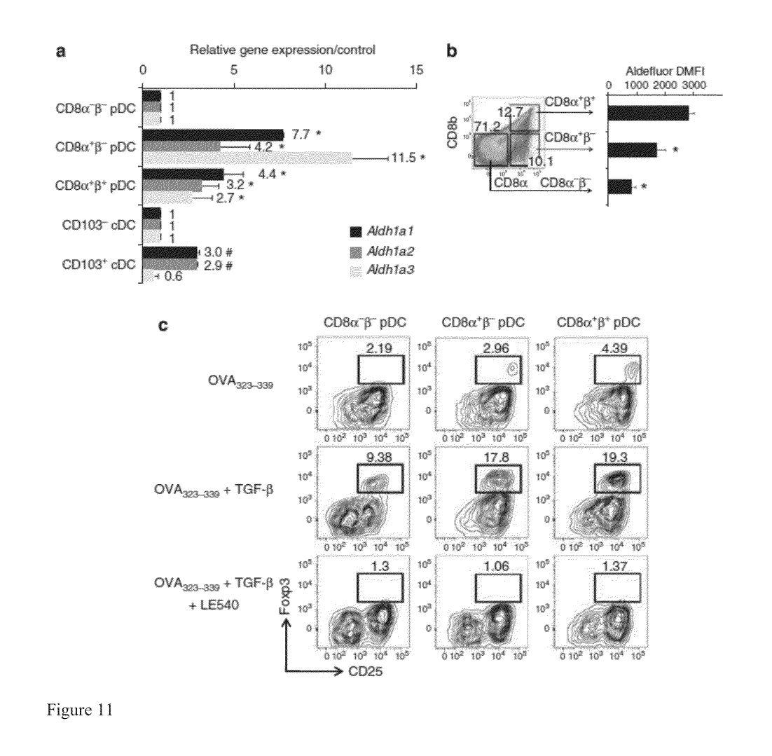

The induction of Treg cells in vivo by tolerogenic DCs has previously been demonstrated to be regulated by TGF-β and retinoid acid²⁰. To test the role of retinoic acid, we analyzed the gene expression of the aldehyde dehydrogenase enzymes (RALDH) that catalyze one step of the conversion of retinol into retinoic acid. We determined that Aldhala1, Aldhala2, and Aldhala3, three genes encoding RALDH1, RALDH2, and RALDH3 enzymes, respectively, were upregulated in the tolerogenic CD8α⁺β⁻ or CD8α⁺β⁺ pDC subsets (FIG. 11a). As a control we tested simultaneously the expression of these genes in CD103⁺ cDCs from mesenteric lymph nodes that have previously been demonstrated to induce Foxp3⁺ Treg cells in a retinoic acid-dependent manner^{21, 22}. In accordance with previous reports, CD103⁺ cDCs expressed high levels of Aldhala1 and Aldahala2 compared with CD103⁻ cDCs but did not express Aldhala3 in contrast to the pDC, subsets described herein (FIG. 11a). To demonstrate the functional activity of RALDH in pDCs, we used the fluorescent RALDH substrate, Aldefluor, in a flow cytometry assay, as has been demonstrated previously^{23,24}. In agreement with the hierarchy of Treg induction capacity in vivo and Aldha genes expression, CD8α⁺β⁺ pDC demonstrated the highest RALDH activity and CD8α⁻β⁻ the lowest (FIG. 7b)). We next tested the requirements for TGF-β and retinoic acid in the conversion of naïve T cells into Treg cells by pDC subsets in vitro. We cultured pDC subsets with naïve OVA-specific CD4⁺ T cells in the presence or absence of TGF-β or TGF-β and RALDH inhibitor (LE540). We observed that in the presence of TGF-β, CD8α⁺β⁺ and CD8α⁺β⁻ pDCs are more efficient in converting naïve CD4+ T cells into Foxp3-expressing Treg cells compared with CD8α⁻β⁻ pDCs (FIG. 11c). In addition, the presence of an RALDH inhibitor completely blocked the conversion of naïve CD4⁺ T cells by CD8α⁺β⁺ and CD8α⁺β⁻ pDCs (FIG. 11c). Altogether, these results demonstrate that CD8α⁺β⁻ pDCs and in particular CD8α⁺β⁺ pDCs strongly support the development of Foxp3⁺ CD4⁺ Treg cells in TGF-13 and retinoic acid dependent manner.

Accordingly, expression of RALDH may be considered a biomarker for tolerogenic antigen presenting cells.

### Translation of pDC Subsets to Human

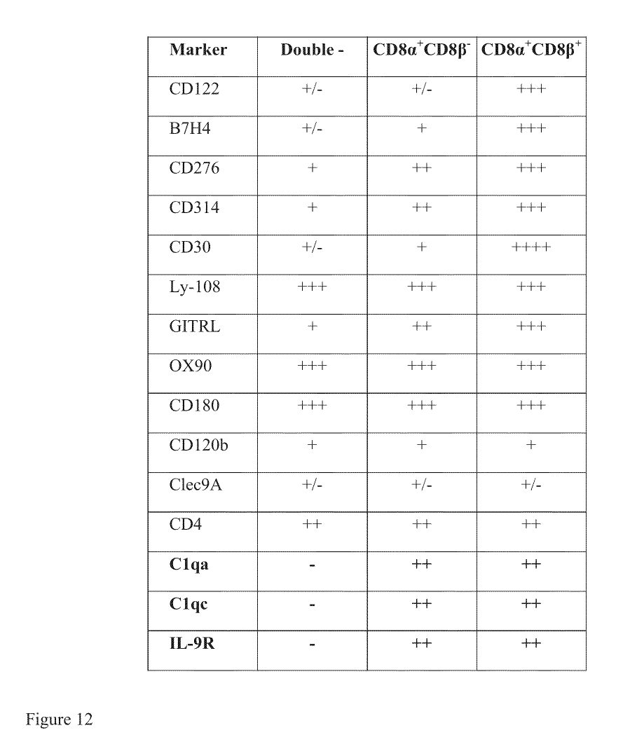

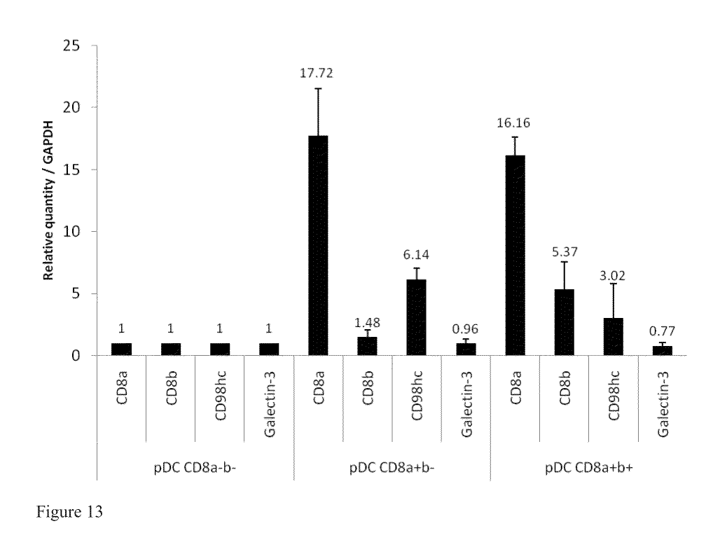

Human pDCs do not express CD8α and CD8β. To uncover human pDCs that share the same tolerogenic properties, we examined the expression patterns of surface markers in mice pDCs. In particular, we performed RNA differential studies and identified several other markers on pDC subsets in mice that can be used to identify tolerogenic pDCs in human. Here we have further discovered that C1qa, C1qc and IL-9R characteristic biomarker for human pDCs (FIGS. 12 and 13).

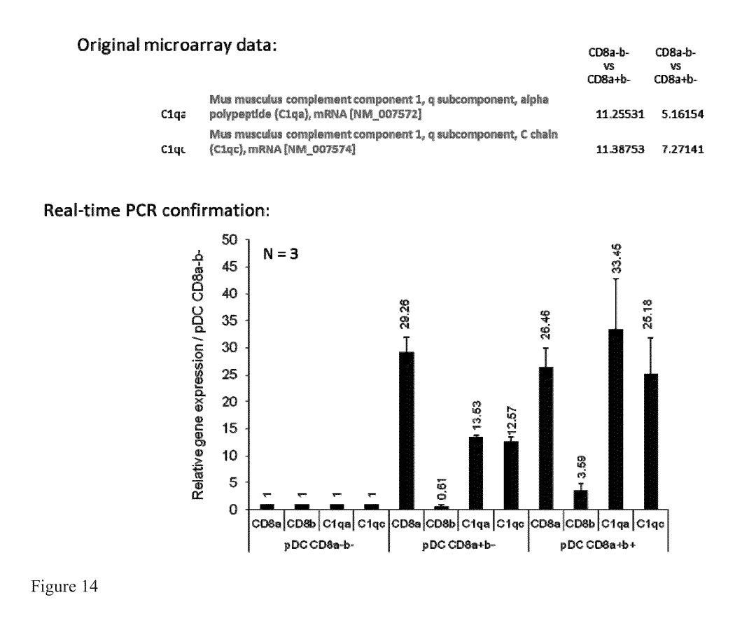

As illustrated in FIG. 13, Galectin-3 and its receptor CD98hc are co-expressed with the tolerogenic pDCs. Similarly, both C1qa and C1qc are found to be up-regulated significantly in tolerogenic pDCs (FIG. 14).

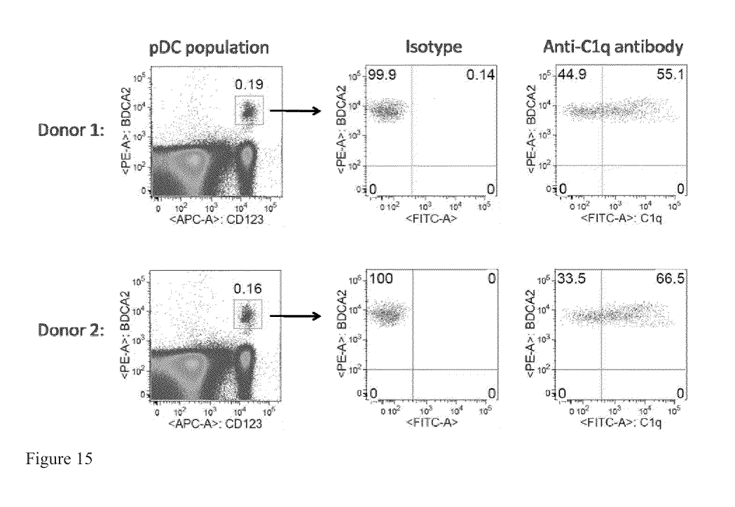

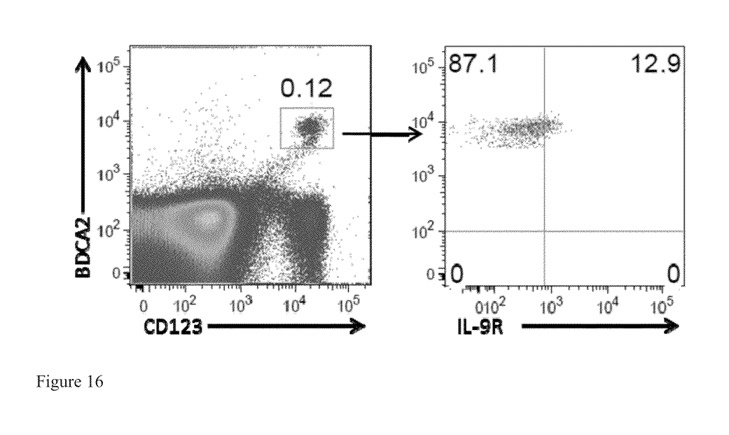

Using C1q-specific antibodies that recognize both C1qa and C1qc (FIG. 15), we were able to isolate tolerogenic pDCs from human peripheral blood mononuclear cells (PBMCs). Similarly, pDCs isolated using anti-IL-9R also are found to be tolerogenic (FIG. 16).

Thus, we have demonstrated here that C1qa, C1qc, and IL-9R are biomarkers for tolerogenic pDCs in human.

### EXPERIMENTAL METHODS

Mice.

Female BALB/c ByJ mice (6 to 8 weeks old) were purchased from The Jackson Laboratory (Bar Harbor, Me.). All mice were maintained in a pathogen-free mouse colony at the Keck School of Medicine (University of Southern California) under protocols approved by the Institutional Animal Care and Use Committee.

Flow Cytometry.

Cells were pre-incubated with anti-Fc receptor mAb 2.402 as well as normal rat serum, and washed before staining. Subsets of dendritic cells were identified using various antibody combinations including Anti-B220 APC-Cy7 (RA3-6B2), anti-CD40 FITC (3/23), anti-CD80 (16-10A1), anti-CD86 PerCP-Cy5.5 (GL1), anti-IA/1E (M5/114.15.2), anti-Ly6C PerCP-Cy5.5 (HK1.4), anti-CD8α PE-Cy7 (53-6.7), anti-CD8β APC (H35-17.2 or 53-5.8), anti-CD3 PerCP-Cy5.5 (145-2C11, all from BD Biosciences, San Jose, Calif.), Siglec-H (eBio440c), anti-CD11c eFluro450 (N418, both from eBioscience, San Diego, Calif.), anti-BST2 PE (120G8.04, Imgenex, San Diego, Calif.) and Ly49Q (2E6, MBL International, Woburn, Mass.). The cells were washed 3 times with cold PBS+2% FCS and were analyzed on the FACS Canto II 8 color flow cytometer (BD Biosciences). The data were analyzed using the FlowJo 6.2 software (Tree Star, Ashland, Oreg.).

Plasmacytoid DC Isolation and Cells Sorting.

To prepare single cell suspension, lymph nodes were digested with 1.6 mg/ml collagenase (CLS4, Worthington Biochemicals, Lakewood N.J.) and 0.1% DNAse I (Fraction IX, Sigma, St. Louis, Mo.) at 37° C. on an orbital shaker for 30 minutes, and for an additional 30 minutes after passing it multiple times through an 18 gauge needle. For in vivo expansion of DCs, 5×10⁶ Flt3Ligand-secreting cells were subcutaneously injected in BALB/c mice. After 14 days, lymph nodes were harvested and processed as described above. To isolate pDCs, cells were labeled with anti-mPDCA-1 microbeads (Miltenyi, Auburn, Calif.) and then positively sorted by AutoMACS according to the manufacturer's instruction. Purity of pDCs was always more than 95%. Plasmacytoid DCs were identified based on their expression of CD11c and BST2; CD8α⁻β⁻, CD8α⁺β⁻ and CD8α⁺β⁺ pDC subsets were separated using a FACS ARIA III cell sorter (BD Biosciences).

Sensitization and Tolerance Models; Measurement of Airway Responsiveness.

CD8α⁻β⁻, CD8α⁺β⁻ and CD8α⁺β⁺ purified pDCs were isolated from lymph nodes of BALB/mice treated with Flt3L-expressing cells. After cell sorting, purified pDCs were loaded with OVA (100 μg/ml) for 4 hours at 37° C. Cells were subsequently washed two times and resuspended in cold saline solution. For the sensitization model, 2×10⁵ cells (pDCs or Bone marrow-derived DCs) were adoptively transferred by intravenous injection through the tail vein. Seven days after the transfer, mice were challenged on three consecutive days by intranasal administration of OVA (50 μg in PBS). For the tolerance model, OVA-loaded pDC subsets were adoptively transferred 7 days prior intraperitoneal injection of OVA (50 μg) in aluminum hydroxide (Alum, 2 mg) and subsequently recipients were challenged intranasally with 3 consecutive doses of OVA (50 μg in PBS) on days 14, 15 and 16. Airway hyperesponsiveness (AHR) responses was subsequently assessed by methacholine-induced airflow obstruction in conscious mice placed in a whole-body plethysmograph (Buxco Electronics, Troy, N.Y.) as described before or by invasive measurement of airway resistance, in which anesthetized and tracheostomized mice were mechanically ventilated. Briefly, Aerosolized methacholine was administered in increasing concentrations of methacholine (0, 2.5, 5 and 10 μg/ml) and we continuously computed the lungs resistance and dynamic compliance by fitting flow, volume, and pressure to an equation of motion. AHR was measured at 24 hours after the last intranasal challenge.

Lungs Histology.

Transcardial perfusion of lungs was performed with cold PBS and subsequently lungs were fixed for histology with 4% paraformaldehyde buffered in PBS. After fixation, the lungs were embedded in paraffin, cut into 4-1 μm sections, and stained with hematoxylin and eosin (H&E) and periodic-acid Schiff (PAS). Histology pictures were acquired using a DFC290 Leica camera (Leica Microsystems, Bannockburn, Ill.).

Confocal Microscopy.

Plasmacytoid DCs were sorted as described above and cells were stained for surface markers with the following antibodies: anti-CD8α Cy5 (53-6.7), anti-CD8β TRITC (H35-17.2, all from eBioscience) and either anti-IA/IE (M5/114.15.2), anti-CD11c (HL3, all from BD Bioscience) or anti-BST2 (mPDCA1, Miltenyi) antibodies conjugated to FITC. Cells were subsequently fixed and permeabilized using the BD Fix/Perm solution. Nucleuses were labeled with Hoescht for 10 minutes. Washed cells were mounted onto slides in Vectashield mounting medium (Vector Laboratories, Burlingame, Calif.). Images were acquired with a Nikon Eclipse Ti confocal microscope (Nikon, Instruments, Melville, N.Y.) and a 100× oil objective associated to the Nikon EC-Z1 software.

In Vitro Culture.

Sorted subpopulation of pDCs were cultured for 24 hours in the presence of CpG 1826 (1 μM, Invivogen, San Diego, Calif.), R848 (10 μg/ml, Alexis Biochemicals, San Diego, Calif.), LPS (10 μg/ml, Invivogen) or medium only. Supernatants were then harvested for further measurement of cytokine production by ELISA for IFN-α (PBL Interferon Source, Piscataway, N.J.) and IL-10 (eBioscience). Sorted CD8α⁻β⁻ pDCs, CD8α⁺β⁻ pDCs and CD8α⁺β⁺ pDCs were co-cultured with CD4⁺ T cells isolated from DO11.10 mice at a 1:10 ratio (1×10⁴ pDCs/1×10⁵ T cells) in a 96-well round bottom plate. Medium was supplemented with OVA peptide (OVA₃₂₃₋₃₃₉, 1 μg/ml, Peptide International, Louisville, Ky.), TGF-β (1 ng/ml, eBiosecience), anti-IL-12 (C17.8), anti-IL-4 (11.B11), anti-IFN-γ (XMG1.2) and anti-IL-6 (MP5-20F3) (all antibodies at 10 μg/ml and purchased from Bioxcell, West Lebanon, N.H.). After three days of culture, cells were harvested, washed and stained to assess Foxp3 expression using the FJK-16s (eBioscience) and the Foxp3 Staining Buffet Set (eBioscience) according to the manufacturer's instructions. Alternatively, cells were pulsed with tritiated thymidine (1 μCi per well) for 18 hours and cell proliferation was evaluated using a beta-counter (Beckman Coulter, Brea, Calif.) as described earlier.

Quantitative Real-Time PCR and Microarray.

Total RNA was extracted from sorted subtypes of pDCs using the RNAasy mini kit (Qiagen) and cDNAs were generated with the High Capacity cDNA Reverse Transcription Kit (Applied Biosystems) according to the manufacturer's recommendations. Quantification of mRNA levels was carried out by quantitative real-time PCR on a CFX96 thermal cycler (Bio-Rad, Hercules, Calif.) with predesigned Taqman gene expression assays for (β-actin: Mm0060732_m1, CD8α: Mm01182108_m1, CD813: Mm00438116_m1, CD98hc: Mm00500521 ml; Applied Biosystems, Foster City, Calif.) and reagents, as per manufacturer's instructions. Microarray processing was performed using the mouse PIQR immunology microarray service from Miltenyi Biotech (Bergisch-Gladbach, Germany).

Analysis of RALDH Activity by Flow Cytometry.

The activity of RALDH enzymes was determined using the Aldefluor staining kit (StemCell Technologies, Vancouver, BC, Canada). pDCs were isolated from pooled peripheral lymph nodes, and incubated for 45 min at 37° C. in the presence of different dilution of BODIPY-aminoacetaldehyde diethyl acetal (Aldefluor substrate) with or without RALDH inhibitor DEAB. Cells were subsequently stained for mPDCA1, CD11c, CD8α and CD8β and analyzed by flow cytometry.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

The entire disclosure of each reference cited herein or listed below is relied upon and incorporated by reference herein.
1. Coquerelle, C. & Moser, M. D C subsets in positive and negative regulation of immunity. Immunol Rev 234, 317-334.
2. Medzhitov, R. & Janeway, C. A., Jr. Decoding the patterns of self and nonself by the innate immune system. Science 296, 298-300 (2002).
3. Palucka, K., Banchereau, J. & Mellman, I. Designing vaccines based on biology of human dendritic cell subsets. Immunity 33, 464-478.
4. Pulendran, B., Tang, H. & Manicassamy, S. Programming dendritic cells to induce T(H)2 and tolerogenic responses. Nat Immunol 11, 647-655.
5. Akbari, O., Stock, P., DeKruyff, R. H. & Umetsu, D. T. Mucosal tolerance and immunity: regulating the development of allergic disease and asthma. Int Arch Allergy Immunol 130, 108-118 (2003).
6. Lambrecht, B. N. & Hammad, H. The role of dendritic and epithelial cells as master regulators of allergic airway inflammation. Lancet 376, 835-843.
7. Lloyd, C. M. & Murdoch, J. R. Tolerizing allergic responses in the lung. Mucosal Immunol 3, 334-344.
8. Siddiqui, K. R. & Powrie, F. CD103+ GALT DCs promote Foxp3+ regulatory T cells. Mucosal Immunol 1 Suppl 1, S34-38 (2008).
9. Coombes, J. L. et al. A functionally specialized population of mucosal CD103+ DCs induces Foxp3+ regulatory T cells via a TGF-beta and retinoic acid-dependent mechanism. J Exp Med 204, 1757-1764 (2007).
10. Akbari, O., DeKruyff, R. H. & Umetsu, D. T. Pulmonary dendritic cells producing IL-10 mediate tolerance induced by respiratory exposure to antigen. Nat Immunol 2, 725-731 (2001).
11. de Heer, H. J. et al. Essential role of lung plasmacytoid dendritic cells in preventing asthmatic reactions to harmless inhaled antigen. J Exp Med 200, 89-98 (2004).
12. Oriss, T. B. et al. Dynamics of dendritic cell phenotype and interactions with CD4+ T cells in airway inflammation and tolerance. J Immunol 174, 854-863 (2005).
13. Hammad, H. et al. Inflammatory dendritic cells—not basophils—are necessary and sufficient for induction of Th2 immunity to inhaled house dust mite allergen. J Exp Med 207, 2097-2111.
14. Wakkach, A. et al. Characterization of dendritic cells that induce tolerance and T regulatory 1 cell differentiation in vivo. Immunity 18, 605-617 (2003).
15. Battaglia, M., Gregori, S., Bacchetta, R. & Roncarolo, M. G. Tr1 cells: from discovery to their clinical application. Semin Immunol 18, 120-127 (2006).
16. Adorini, L. & Penna, G. Induction of tolerogenic dendritic cells by vitamin D receptor agonists. Handb Exp Pharmacol, 251-273 (2009).
17. Wang, Z. et al. Use of the inhibitory effect of apoptotic cells on dendritic cells for graft survival via T-cell deletion and regulatory T cells. Am J Transplant 6, 1297-1311 (2006).
18. Ilarregui, J. M. et al. Tolerogenic signals delivered by dendritic cells to T cells through a galectin-1-driven immunoregulatory circuit involving interleukin 27 and interleukin 10. Nat Immunol 10, 981-991 (2009).
19. Hu, J. & Wan, Y. Tolerogenic dendritic cells and their potential applications. Immunology.
20. Mucida, D. et al. Reciprocal TH17 and regulatory T cell differentiation mediated by retinoic acid. Science 317, 256-260 (2007).
21. Coombes, J. L. et al. A functionally specialized population of mucosal CD103+DCs induces Foxp3+ regulatory T cells via a TGF-beta and retinoic acid-dependent mechanism. J. Exp. Med. 204, 1757-1764 (2007).
22. Sun, C. M. et al. Small intestine lamina propria dendritic cells promote de novo generation of Foxp3 T reg cells via retinoic acid. J. Exp. Med. 204, 1775-1785 (2007).
23. Guilliams, M. et al, Skin-draining lymph nodes contain dermis-derived CD103(−) dendritic cells that constitutively produce retinoic acid and induce Foxp3(+) regulatory T cells. Blood 115, 1958-1968 (2010).
24. Stock, A., Booth, S. & Cerundolo, V. Prostaglandin E2 suppresses the differentiation of retinoic acid-producing dendritic cells in mice and humans. J. Exp. Med 208, 761-773 (2011).

What is claimed is:

1. One or more isolated plasmacytoid dendritic cells (pDCs) selected from the group consisting of CD8α⁻β⁻, CD8α⁺β⁻, CD8α⁺β⁺, C1qa⁺c⁺, IL-9R⁺ and a combination of any two of CD8α⁻β⁻, CD8α⁺β⁻, CD8α⁺β⁺.

2. The isolated pDCs according to claim 1, wherein said pDCs are CD8α⁻β⁻.

3. The isolated pDCs according to claim 1, wherein said pDCs are CD8α⁺β⁺.

4. The isolated pDCs according to claim 1, wherein said pDCs are CD8α⁺β⁻.

5. The isolated pDCs according to claim 1, wherein said pDCs are a combination of CD8α⁺β⁻ and CD84α⁺β⁺.

6. The isolated pDCs according to claim 1, wherein said pDCs are human C1qa⁺c⁺ or IL-9R⁺.

7. A composition, comprising:
a tolerogenic or immunogenic antigen presenting cell; and
a carrier.

8. The composition of claim 7, wherein said antigen presenting cell is a tolerogenic pDC selected from CD8α⁺β⁻, CD8α⁺β⁺, a combination of CD8α⁺β⁻, CD8α⁺β⁺, a human C1qa⁺c⁺, and a human IL-9R⁺.

9. The composition of claim 8, further comprising TGF-β, galectin-3, or both.

10. The composition of claim 7, wherein said antigen presenting cell is CD8α⁻β⁻.

11. The composition of claim 10, further comprising an inhibitor of RALDH.

12. The composition of claim 7, wherein said antigen presenting cell is pre-incubated with an antigen.

13. A method for isolating a pDC having tolerogenic property, comprising:
enriching pDCs from a source sample; and
sorting the enriched pDCs according to their CD8 surface marker subtypes.

14. A method for preventing or treating immune-hyper-reactivity in a subject, comprising:
administering to said subject an effective amount of a composition according to claim 7.

15. The method of claim 14, wherein said immune-hyper-reactivity is inflammation, allergy, or asthma.

16. A method for inducing conversion of naïve CD4⁺ T cells into Foxp3+ regulatory T cells, comprising:
brining a tolerogenic antigen presenting cell into fluid communication with a naïve CD4⁺ T cell.

17. The method of claim 16, wherein said tolerogenic antigen presenting cell is a tolerogenic pDC selected from the group consisting of CD8α⁻, CD8α⁺β⁺, and a combination thereof.

18. The method of claim 16, wherein said bringing step is done in the presence of TGF-β, Galectin-3, or both.

19. A method for modulating immune response in a subject who is suffering from an immune hyper-reactivity disorder against an antigen or is in need of boosting an immune response against an antigen, said method comprising:
administering an effective amount of a pharmaceutical composition to said subject, wherein:
said pharmaceutical composition is one comprising a tolerogenic antigen presenting cell pre-loaded with the antigen when said subject is suffering from an immune hyper-reactivity disorder, or
said pharmaceutical composition is one comprising an immunogenic antigen presenting cell pre-loaded with the antigen when said subject is in need of boosting an immune response against the antigen.

20. The method of claim 19, wherein said subject is one suffering from an immune hyper-reactivity, and said antigen presenting cell is a tolerogenic pDC.

21. The method of claim 19, wherein said subject is one in need of boosting an immune response, and said antigen presenting cell is an immunogenic pDC.

22. A method for identifying a tolerogenic antigen presenting cell, comprising:
determining the expression levels for RALDH1, RALDH2, and RALDH3 in the antigen presenting cell; and
designating the antigen presenting cell as tolerogenic if all of RALDH1, RALDH2, and RALDH3 are up-regulated compare to a predetermined reference.
